(19) United States
(12) Patent Application Publication
(10) Pub. No.: US 2026/0256481 A1
(43) Pub. Date: Sep. 3, 2026

(54) MEDICAL DEVICE

(71) Applicant: ASAHI INTECC CO., LTD., Seto-shi (JP)

(72) Inventor: Hirokatsu TAKEMOTO, Seto-shi (JP)

(73) Assignee: ASAHI INTECC CO., LTD., Seto-shi (JP)

(21) Appl. No.: 19/353,071

(22) Filed: Oct. 8, 2025

(30) Foreign Application Priority Data
Feb. 28, 2025 (JP) 2025-030833

(51) Int. Cl. A61B 17/22; A61M 25/09

(52) U.S. Cl. CPC A61B 17/22; A61M 25/09; A61B 2017/22044; A61B 2017/22094; A61M 2025/09083; A61M 2025/09175; A61M 2210/12

(57) ABSTRACT

A medical device includes an elongated main body part and a leading portion connected to a distal end of the main body part. The leading portion includes a rod body that can enter a lesion. A distal end portion of the main body part extends in a first direction. A distal end portion of the rod body extends in a second direction different from the first direction.

### CROSS-REFEENCE TO RELATED APPLICATIONS

This application claims the benefit of JP 2025-030833, filed February 28, 2025. The disclosure of the prior application is hereby incorporated by reference herein in its entirety.

### TECHNICAL FIELD

A technology as disclosed herein relates to a medical device.

### BACKGROUND

A known medical device includes an elongated portion including a shaft, a coil, and the like, and a probe extending distally from a distal end of the elongated portion. The elongated portion is bent (for example, see Patent Document 1).

### PRIOR ART DOCUMENT

### Patent Document

Patent Document 1: JP 2012-505026 A

### SUMMARY

### Technical Problem

When the medical device receives an external force in a living body lumen such as a blood vessel, the medical device is easily deformed from a shape before receiving the external force. That is, the medical device has a low shape memory property.

The present specification discloses a technology that can solve the above-described problem.

### Solution to Problem

The technology as disclosed herein can be achieved, for example, as the following aspect.

A medical device as disclosed herein includes: an elongated main body part; and a leading portion connected to a distal end of the main body part. The leading portion includes a rod body that can enter a lesion. A distal end portion of the main body part extends in a first direction. A distal end portion of the rod body extends in a second direction different from the first direction.

### BRIEF DESCRIPTION OF DRAWINGS

FIG. 1 is an explanatory view schematically illustrating a configuration of a guide wire according to a first embodiment.

FIG. 2 is an explanatory view schematically illustrating a configuration of the guide wire according to the first embodiment.

FIG. 3 is an explanatory view schematically illustrating a configuration of the guide wire according to the first embodiment.

FIG. 4 is an explanatory view schematically illustrating a configuration of the guide wire according to the first embodiment.

FIG. 5 is an explanatory view schematically illustrating a configuration of the guide wire according to the first embodiment.

FIG. 6 is a flowchart illustrating an example of a treatment method using the guide wire.

FIG. 7 is an explanatory view illustrating an example of the treatment method using the guide wire.

FIG. 8 is an explanatory view illustrating an example of the treatment method using the guide wire.

FIG. 9 is an explanatory view schematically illustrating a configuration of a guide wire according to a second embodiment.

FIG. 10 is an explanatory view schematically illustrating a configuration of a guide wire according to a third embodiment.

FIG. 11 is an explanatory view schematically illustrating a configuration of a guide wire according to a first modification.

FIG. 12 is an explanatory view schematically illustrating a configuration of a guide wire according to a second modification.

FIG. 13 is an explanatory view schematically illustrating a configuration of a guide wire according to a third modification.

FIG. 14 is an explanatory view schematically illustrating a configuration of a guide wire according to a fourth modification.

### DETAILED DESCRIPTION

### First Embodiment

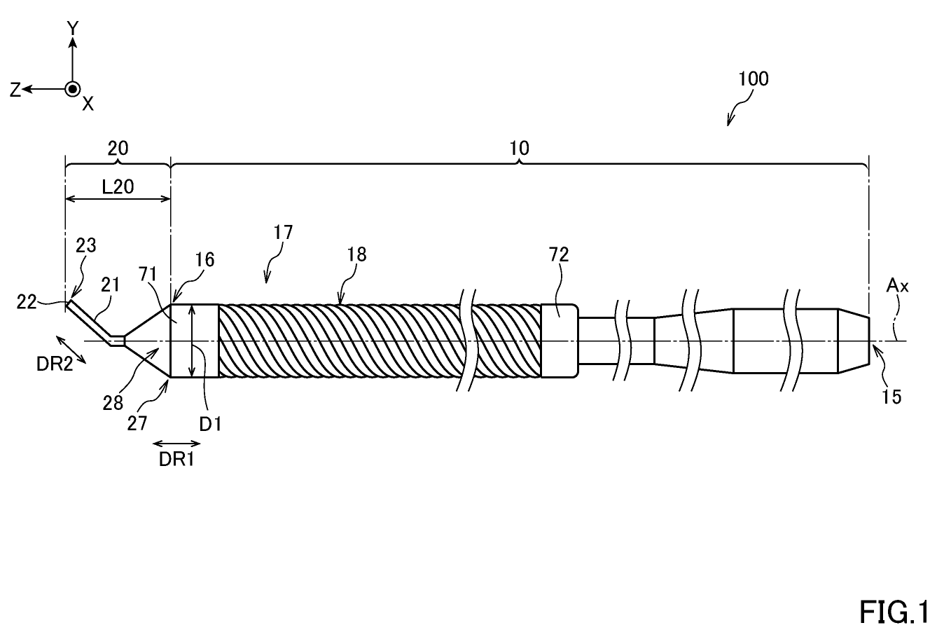

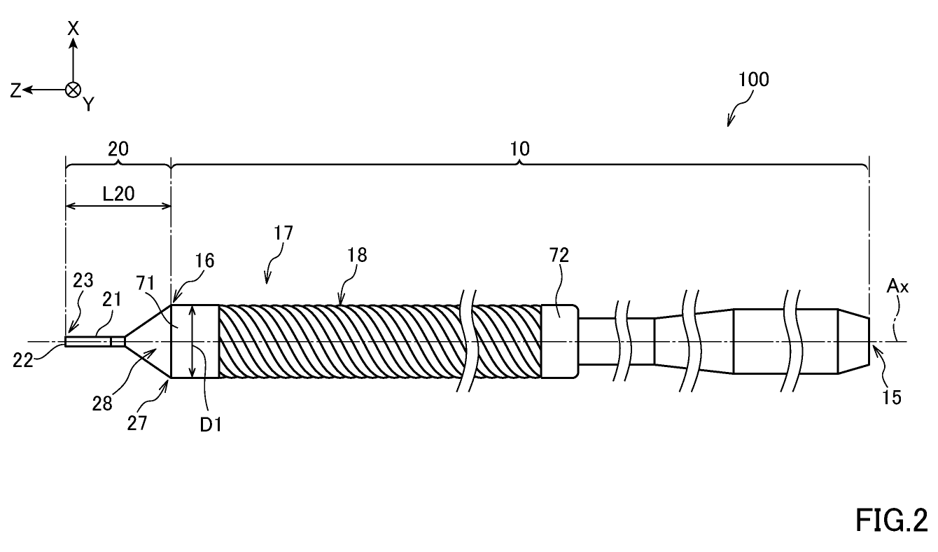

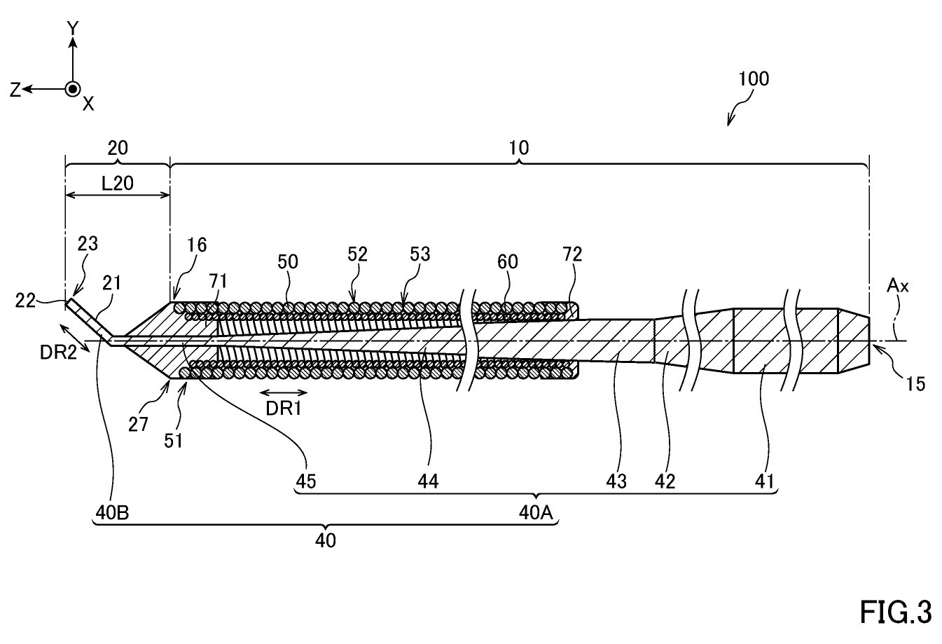

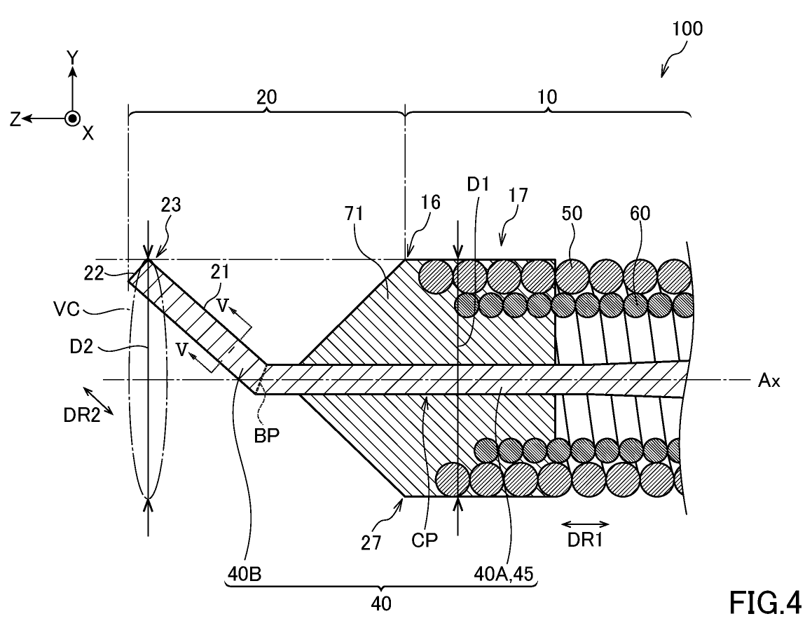

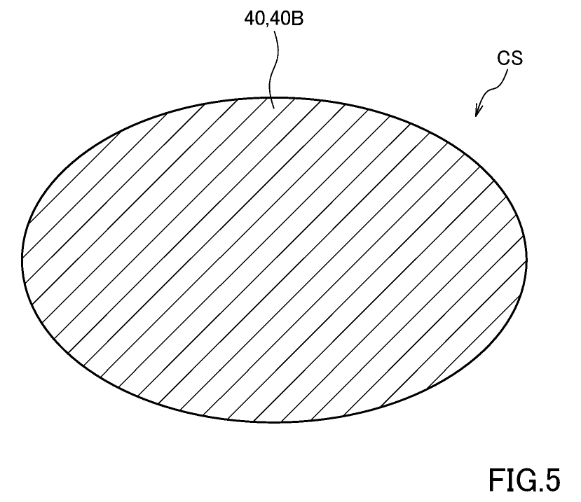

FIGS. 1 to 5 are explanatory views schematically illustrating a configuration of a guide wire 100 according to a first embodiment. In each drawing, XYZ axes orthogonal to each other for specifying a direction are illustrated. FIG. 1 illustrates an external appearance of the guide wire 100 as viewed in an X-axis direction. FIG. 2 illustrates an external appearance of the guide wire 100 as viewed in a Y-axis direction. FIG. 3 illustrates a YZ longitudinal section of the guide wire 100. FIG. 4 illustrates a YZ longitudinal section of a distal end portion of the guide wire 100. FIG. 5 illustrates a transverse section CS of a core shaft 40 to be described below at a position V-V in FIG. 4. In the guide wire 100, the positive side of the Z-axis is a distal end side (far side) to be inserted into a body. In the guide wire 100, the negative side of the Z-axis is a proximal end side (near side) to be operated by a professional. In each drawing, a part of the guide wire 100 may be omitted. FIGS. 1 to 4 illustrate a state where the guide wire 100 is in a linear shape parallel to the Z-axis. The guide wire 100 has sufficient flexibility to be curved.

In the present specification, regarding the guide wire 100 and each component thereof, an end on the distal end side is referred to as a “distal end”, the distal end and the vicinity thereof are referred to as a “distal end portion”, an end on the proximal end side is referred to as a “proximal end”, and the proximal end and the vicinity thereof are referred to as a “proximal end portion”. A transverse section of the guide wire 100 and each component thereof is meant to be a cross section orthogonal to a longitudinal direction. A longitudinal section of the guide wire 100 and each component thereof is meant to be a cross section parallel to a center axis in the longitudinal direction. In the guide wire 100 and each component thereof, a direction orthogonal to the longitudinal direction is referred to as a “radial direction”. An outer diameter of the guide wire 100 and each component thereof is meant to be a width along the radial direction.

The guide wire 100 is an elongated medical device to be inserted into a living body lumen such as a blood vessel. An entire length of the guide wire 100 is, for example, not less than 1000mm and not more than 3000mm. The guide wire 100 is an example of the medical device.

The guide wire 100 includes a main body part 10 and a leading portion 20.

The main body part 10 is an elongated portion extending along a center axis Ax. A proximal end 15 of the main body part 10 coincides with the proximal end of the guide wire 100. A spiral groove 18 is formed on an outer peripheral surface 17 of a distal end portion of the main body part 10. The distal end portion of the main body part 10 extends in a first direction DR1. The first direction DR1 is a direction parallel to a Z-axis direction.

The leading portion 20 is connected to a distal end 16 of the main body part 10. A proximal end 27 of the leading portion 20 is connected to the distal end 16 of the main body part 10. The leading portion 20 has a rod body 21 that enters a lesion. The leading portion 20 can be expressed as a guiding portion, a drilling portion, a fracturing portion, a peeling portion, an advancing portion, a peeler, a shaver, or the like. A distal end 22 of the rod body 21 coincides with a distal end 23 of the leading portion 20. The distal end 22 of the rod body 21 coincides with the distal end of the guide wire 100. The distal end 23 of the leading portion 20 is a planar surface parallel to a direction orthogonal to a long axis direction (a second direction DR2) of the leading portion 20. The distal end 22 of the rod body 21 is a planar surface parallel to a direction orthogonal to a long axis direction (a second direction DR2) of the rod body 21. The leading portion 20 enters the lesion while rotating around the center axis Ax of the main body part 10. The entry of the leading portion 20 into the lesion can be expressed as crossing/passing through the lesion, drilling the lesion, fracturing (peeling) the lesion, pushing through the lesion, advancing into the lesion, and the like.

The rod body 21 of the leading portion 20 extends linearly. A distal end portion of the rod body 21 extends in the second direction DR2. The second direction DR2 is parallel to the YZ plane and intersects with both the Z-axis direction and the Y-axis direction. The second direction DR2 is a direction inclined at a predetermined angle with respect to the first direction DR1. The predetermined angle is, for example, not less than 10 degrees and not more than 70 degrees. The predetermined angle may be not less than 15 degrees and not more than 60 degrees or may be not less than 30 degrees and not more than 50 degrees. An outer diameter of the transverse section of the rod body 21 in the leading portion 20 is smaller than an outer diameter of the transverse section of the main body part 10. A length L20 of the leading portion 20 along the Z-axis direction is, for example, not less than 0.2mm and not more than 2.0mm. The length L20 of the leading portion 20 may be not less than 0.3mm and not more than 1.5mm or may be not less than 0.4mm and not more than 1.0mm.

As illustrated in FIG. 4, the distal end 22 of the rod body 21 is located at the same position as the outer peripheral surface 17 of the main body part 10 in the radial direction of the guide wire 100. When a locus traced by the distal end 22 of the rod body 21 in a case where the guide wire 100 is rotated around the center axis Ax is defined as a virtual circle VC, a diameter D2 of the virtual circle VC is equal to a diameter D1 of the main body part 10. The diameter D1 is, in particular, a diameter of the main body part 10 at the distal end 16 of the main body part 10.

As illustrated in FIG. 3, the guide wire 100 includes a core shaft 40, a first coil 50, and a second coil 60.

The core shaft 40 is a linear member. A part of the core shaft 40 is covered by each of the first coil 50 and the second coil 60. The core shaft 40 includes a first portion 40A and a second portion 40B. FIG. 4 illustrates a boundary position BP between the first portion 40A and the second portion 40B. The boundary position BP is a distal end of the first portion 40A and a proximal end of the second portion 40B. The main body part 10 includes a part of the core shaft 40 on the proximal end side of the first portion 40A. The leading portion 20 includes a part of the core shaft 40 on the distal end side of the first portion 40A, and the second portion 40B.

The first portion 40A is a portion of the core shaft 40. A proximal end of the first portion 40A is located at a proximal end portion of the main body part 10. The proximal end side of the first portion 40A is a portion to be gripped by a professional. The first portion 40A extends along the center axis Ax parallel to the Z-axis direction.

The first portion 40A includes a large diameter portion 41, a first tapered portion 42, an intermediate diameter portion 43, a second tapered portion 44, and a small diameter portion 45. The large diameter portion 41, the first tapered portion 42, the intermediate diameter portion 43, the second tapered portion 44, and the small diameter portion 45 are arranged in this order from the proximal end side toward the distal end side of the guide wire 100.

The large diameter portion 41 is a rod-shaped portion having a substantially constant outer diameter. The outer diameter (maximum width) of the large diameter portion 41 is, for example, about not less than 0.2mm and not more than 3.0mm. The first tapered portion 42 is a portion whose outer diameter gradually decreases from a boundary with the large diameter portion 41 toward a boundary with the intermediate diameter portion 43. The intermediate diameter portion 43 is a rod-shaped portion having a substantially constant outer diameter that is smaller than the outer diameter of the large diameter portion 41. The second tapered portion 44 is a portion whose outer diameter gradually decreases from the boundary with the intermediate diameter portion 43 toward a boundary with the small diameter portion 45. The small diameter portion 45 is a rod-shaped portion having a substantially constant outer diameter that is smaller than the outer diameter of the intermediate diameter portion 43. The small diameter portion 45 is a distal end portion of the first portion 40A. The small diameter portion 45 extends in the first direction DR1.

The second portion 40B is a portion of the core shaft 40 different from the first portion 40A. The second portion 40B is located on the distal end side with respect to the first portion 40A. The proximal end of the second portion 40B is connected to the distal end of the first portion 40A. A distal end of the second portion 40B is located at a distal end portion of the leading portion 20. The distal end of the second portion 40B is a planar surface parallel to a direction orthogonal to a long axis direction (a second direction DR2) of the second portion 40B. The second portion 40B extends linearly from a boundary position BP with the first portion 40A to the distal end of the second portion 40B. The second portion 40B extends in the second direction DR2. The distal end of the second portion 40B is located at the same position as an outer peripheral surface 52 of the first coil 50 in the radial direction of the guide wire 100. As illustrated in FIG. 5, the transverse section CS of the second portion 40B of the core shaft 40 has a flat shape.

As a material forming the core shaft 40, for example, a metal is used. More specifically, for example, stainless steels (SUS302, SUS304, SUS316, etc.), Ni-Ti alloys, piano wire, and the like are used. The core shaft 40 may be wholly formed of the same material, or individual portions may each be formed of different materials.

The first coil 50 is a cylindrical member in which one or more wires are spirally wound. The main body part 10 includes the first coil 50. The first coil 50 covers the core shaft 40. An outer diameter of the first coil 50 is, for example, not less than 0.1mm and not more than 0.6mm. The outer diameter of the first coil 50 may be not less than 0.2mm and not more than 0.5mm or may be not less than 0.3mm and not more than 0.4mm. The outer diameter of the first coil 50 may be not less than 1.00mm and not more than 2.00mm, may be not less than 1.10mm and not more than 1.65mm, or may be not less than 1.20mm and not more than 1.35mm. In the present embodiment, the outer diameter of the first coil 50 is constant over the entire length of the first coil 50. A spiral groove 53 is formed on the outer peripheral surface 52 of the first coil 50. Due to the presence of the spiral groove 53, the spiral groove 18 is formed on the outer peripheral surface 17 of the main body part 10. A distal end 51 of the first coil 50 coincides with the distal end 16 of the main body part 10. The first coil 50 is an example of a tubular body.

The second coil 60 is a cylindrical member in which one or more wires are spirally wound. The main body part 10 includes the second coil 60. The second coil 60 is located between the core shaft 40 and the first coil 50 in the radial direction of the main body part 10. The second coil 60 covers the core shaft 40. A distal end of the second coil 60 covers a distal end portion of a portion of the core shaft 40 included in the main body part 10. An inner diameter of the second coil 60 is larger than an outer diameter of a portion of the core shaft 40 covered by the second coil 60. The second coil 60 is covered by the first coil 50. An outer diameter of the second coil 60 is smaller than an inner diameter of the first coil 50.

As a material forming the first coil 50 and the second coil 60, for example, a metal is used. More specifically, for example, radiolucent materials such as stainless steels (SUS302, SUS304, SUS316, etc.), Ni-Ti alloys, piano wire, and radiopaque materials such as platinum, gold, tungsten, and any of alloys thereof may be used. The first coil 50 and the second coil 60 may be formed of the same material or may be formed of different materials. Each of the first coil 50 and the second coil 60 may be wholly formed of the same material, or individual portions may each be formed of different materials.

The first coil 50 and the second coil 60 are joined to the core shaft 40 via a distal-end-side bonding material 71 formed at distal end portions of the first coil 50 and the second coil 60 and a proximal-end-side bonding material 72 formed at proximal end portions of the first coil 50 and the second coil 60. The distal-end-side bonding material 71 protrudes from the distal end 51 of the first coil 50 toward the distal end side. The first portion 40A of the core shaft 40 is connected to each of the first coil 50 and the second coil 60 via a portion of the distal-end-side bonding material 71 protruding from the distal end 51 of the first coil 50. In the longitudinal direction of the guide wire 100, the proximal end of the second portion 40B is located on the distal end side with respect to a joint part CP of the core shaft 40 with the first coil 50. The joint part CP is a part of the core shaft 40 and is a section in which the distal-end-side bonding material 71 is attached to the core shaft 40. A boundary position BP between the first portion 40A and the second portion 40B is located on the distal end side of the guide wire 100 with respect to the distal-end-side bonding material 71. As a material for forming the distal-end-side bonding material 71 and the proximal-end-side bonding material 72, for example, a metal solder (Au-Sn alloy, Sn-Ag alloy, Sn-Pb alloy, Pb-Ag alloy, etc.), a brazing material (aluminum alloy braze, silver braze, gold braze, etc.), an adhesive (epoxy-based adhesive, etc.), or the like is used.

The leading portion 20 has a reinforcing portion 28 located at a connection location with the main body part 10. The connection location of the leading portion 20 with the main body part 10 is reinforced by the reinforcing portion 28. In the present embodiment, the reinforcing portion 28 is formed of a portion of the distal-end-side bonding material 71 protruding from the distal end 51 of the first coil 50 toward the distal end side. The reinforcing portion 28 may be formed by a welded portion between the leading portion 20 and the main body part 10.

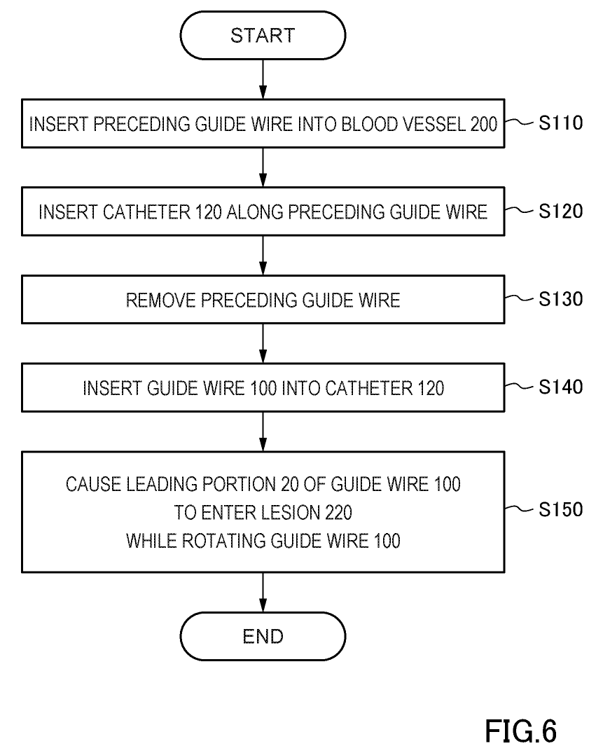

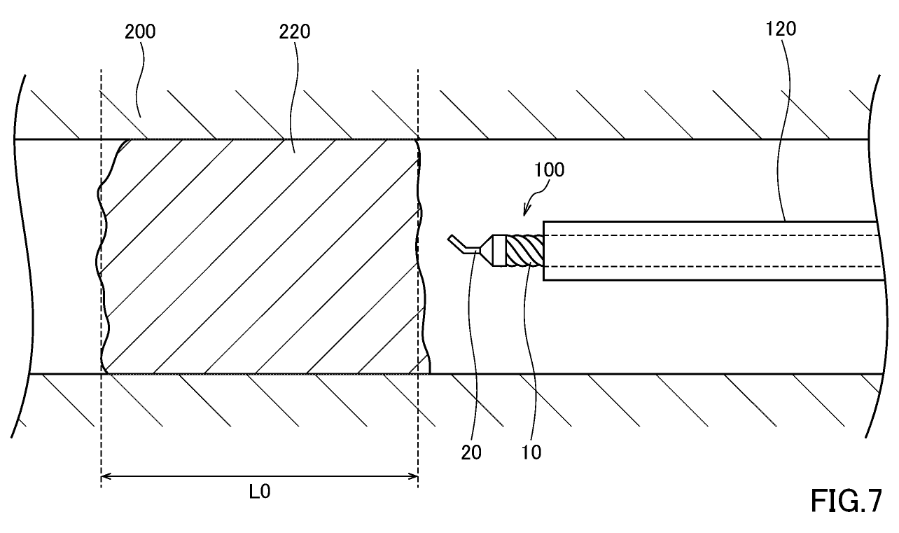

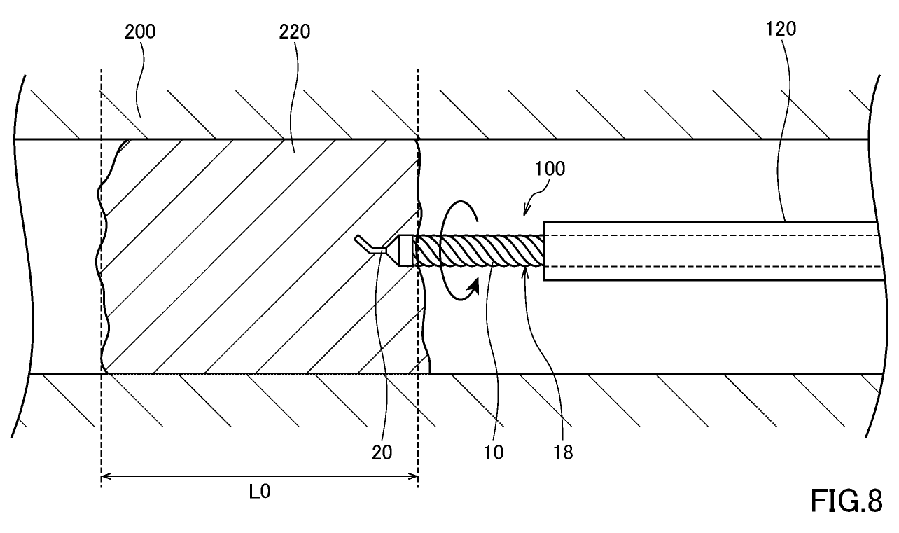

FIG. 6 is a flowchart illustrating an example of a treatment method using the guide wire 100. FIGS. 7 to 8 are explanatory views illustrating an example of the treatment method using the guide wire 100. As illustrated in FIGS. 7 and 8, in the treatment method using the guide wire 100, a professional causes the leading portion 20 of the guide wire 100 to enter a lesion 220 in a blood vessel 200. The lesion 220 is, for example, a highly calcified lesion. The lesion 220 is, for example, a chronic total occlusion lesion. A length L0 of the lesion 220 along an extending direction of the blood vessel 200 is, for example, not less than 100mm and not more than 500mm. The length L0 of the lesion 220 may be not less than 150mm and not more than 450mm, or may be not less than 200mm and not more than 400mm.

First, the professional inserts a preceding guide wire into the blood vessel 200 (S110). Unlike the guide wire 100 according to the present embodiment, the preceding guide wire is a known guide wire that does not have the leading portion 20. The preceding guide wire may be referred to as a workhorse guide wire, a first-choice guide wire, or the like. The professional inserts the preceding guide wire into the blood vessel 200 via a sheath disposed at an arbitrary position on the body surface of a patient. The professional advances the preceding guide wire to a location immediately proximal to the lesion 220 in the blood vessel 200.

Next, the professional inserts a catheter 120 into the blood vessel 200 along the preceding guide wire (S120). The professional advances the catheter 120 (see FIG. 7) to a location immediately proximal to the lesion 220 in the blood vessel 200.

Next, the professional removes the preceding guide wire from the blood vessel 200 (S130). Thereafter, the professional inserts the guide wire 100 into the catheter 120 inserted into the blood vessel 200, with the leading portion 20 at the front (S140, FIG. 7). The professional advances the guide wire 100 to a location immediately proximal to the lesion 220 in the blood vessel 200. When advancing the guide wire 100, the professional may or may not rotate the guide wire 100 around the center axis Ax.

Next, while rotating the guide wire 100, the professional advances the guide wire 100 toward the distal end side, thereby causing the leading portion 20 of the guide wire 100 to enter the lesion 220 (S150, FIG. 8). When the professional grips the proximal end portion of the guide wire 100 and rotates the guide wire 100 around the center axis Ax, the leading portion 20 located at the distal end portion of the guide wire 100 also rotates around the center axis Ax. The leading portion 20, which is rotating within the lesion 220, drills the lesion 220 in a manner that tears apart the lesion 220. When the leading portion 20 passes through the lesion 220, a through-hole having an inner diameter equal to the diameter D2 of the virtual circle VC is formed in the lesion 220. In the present embodiment, in the step of advancing the guide wire 100 (S150), the professional advances the leading portion 20 until the leading portion 20 passes through the lesion 220. The step of advancing the guide wire 100 (S150) is performed in a state where none of other medical devices have passed through the lesion 220.

After the leading portion 20 of the guide wire 100 has passed through the lesion 220, the professional advances a catheter (not illustrated) to the location of the lesion 220 along the guide wire 100. Thereafter, the professional removes the guide wire 100. When removing the guide wire 100, the professional may or may not rotate the guide wire 100 around the center axis Ax.

Thereafter, the professional inserts a guide wire for an adjunctive device (not illustrated) into the blood vessel 200 and advances the guide wire for an adjunctive device until a distal end of the guide wire for an adjunctive device passes through the lesion 220. The professional advances the adjunctive device to the location of the lesion 220 along the guide wire for the adjunctive device. Examples of the adjunctive device include a device for atherectomy, a balloon catheter, a stent, and the like.

As described above, the guide wire 100 of the present embodiment includes the elongated main body part 10, and the leading portion 20 connected to the distal end 16 of the main body part 10, the leading portion 20 having the rod body 21 that enters the lesion 220. The distal end portion of the main body part 10 extends in the first direction DR1. The distal end portion of the rod body 21 extends in the second direction DR2 different from the first direction DR1. According to the guide wire 100 of the present embodiment, since the guide wire 100 is bent on the distal end side with respect to the main body part 10, a shape memory property of the guide wire 100 is improved as compared to, for example, a medical device in which the main body part is bent.

In the guide wire 100 of the present embodiment, the distal end 22 of the rod body 21 is located at the same position as the outer peripheral surface 17 of the main body part 10 in the radial direction of the guide wire 100. According to the guide wire 100 of the present embodiment, since the distal end 22 of the rod body 21 is located at the same position as the outer peripheral surface 17 of the main body part 10, the shape memory property of the guide wire 100 is improved. According to the guide wire 100 of the present embodiment, since the distal end 22 of the rod body 21 is located at the same position as the outer peripheral surface 17 of the main body part 10, a through-hole equal to the diameter D1 of the main body part 10 can be formed in the lesion 220, and passability of the guide wire 100 with respect to the lesion 220 is improved. That is, according to the guide wire 100 of the present embodiment, both the shape memory property and the passability of the guide wire 100 are achieved.

In the guide wire 100 of the present embodiment, the main body part 10 includes a first portion 40A that is a portion of the core shaft 40, the first portion 40A having a distal end portion thereof extending in the first direction DR1, and the leading portion 20 includes a second portion 40B that is a portion of the core shaft 40 different from the first portion 40A, the second portion 40B being connected to the distal end of the first portion 40A and extending in the second direction DR2. According to the guide wire 100 of the present embodiment, the shape memory property of the guide wire 100 is improved.

In the guide wire 100 of the present embodiment, the main body part 10 further includes the first coil 50 that covers the core shaft 40 and that is joined to the core shaft 40. According to the guide wire 100 of the present embodiment, torquability and durability of the guide wire 100 are improved.

In the guide wire 100 of the present embodiment, in the longitudinal direction of the guide wire 100, the proximal end of the second portion 40B is located on the distal end side with respect to the joint part CP of the core shaft 40 with the first coil 50. According to the guide wire 100 of the present embodiment, since the second portion 40B is not joined to the first coil 50, it is difficult to fix an angle between an extending direction of the main body part 10 and an extending direction of the leading portion 20. Therefore, for example, when the guide wire 100 is used in combination with an adjunctive device such as a catheter covering the guide wire 100, the leading portion 20 is prevented from coming into strong contact with the adjunctive device, and a decrease in slidability between the leading portion 20 and the adjunctive device is prevented.

In the guide wire 100 of the present embodiment, the guide wire 100 further includes the second coil 60 located between the core shaft 40 and the first coil 50 in the radial direction of the guide wire 100. According to the guide wire 100 of the present embodiment, the torquability and durability of the guide wire 100 are improved.

In the guide wire 100 of the present embodiment, the transverse section CS of the second portion 40B has a flat shape. According to the guide wire 100 of the present embodiment, the shape memory property of the guide wire 100 is improved.

### Second Embodiment

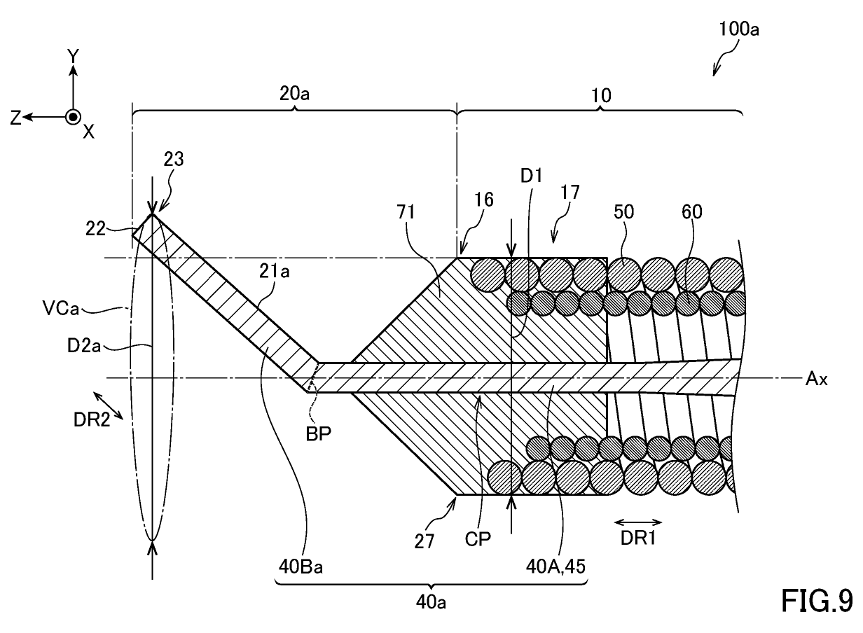

FIG. 9 is an explanatory view schematically illustrating a configuration of a guide wire 100a according to a second embodiment. FIG. 9 illustrates a YZ longitudinal section of a distal end portion of the guide wire 100a. Hereinafter, components of the guide wire 100a according to the second embodiment, which are the same as the components of the guide wire 100 according to the first embodiment, are denoted with the same reference signs, thereby omitting description thereof as appropriate.

In the guide wire 100a of the present embodiment, a distal end 22 of a rod body 21a is located outside an outer peripheral surface 17 of a main body part 10 in a radial direction of the guide wire 100a. When a locus traced by the distal end 22 of the rod body 21a when the guide wire 100a is rotated around a center axis Ax is defined as a virtual circle VCa, a diameter D2a of the virtual circle VCa is larger than a diameter D1 of the main body part 10. In the radial direction of the guide wire 100a, a distal end of a second portion 40Ba of a core shaft 40a is located outside an outer peripheral surface of a first coil 50.

As described above, in the guide wire 100a of the present embodiment, the distal end 22 of the rod body 21a is located outside the outer peripheral surface 17 of the main body part 10 in the radial direction of the guide wire 100a. According to the guide wire 100a of the present embodiment, since the distal end 22 of the rod body 21a is located outside the outer peripheral surface 17 of the main body part 10, a through-hole larger than the diameter D1 of the main body part 10 can be formed in a lesion 220, and passability of the guide wire 100a with respect to the lesion 220 is improved.

### Third Embodiment

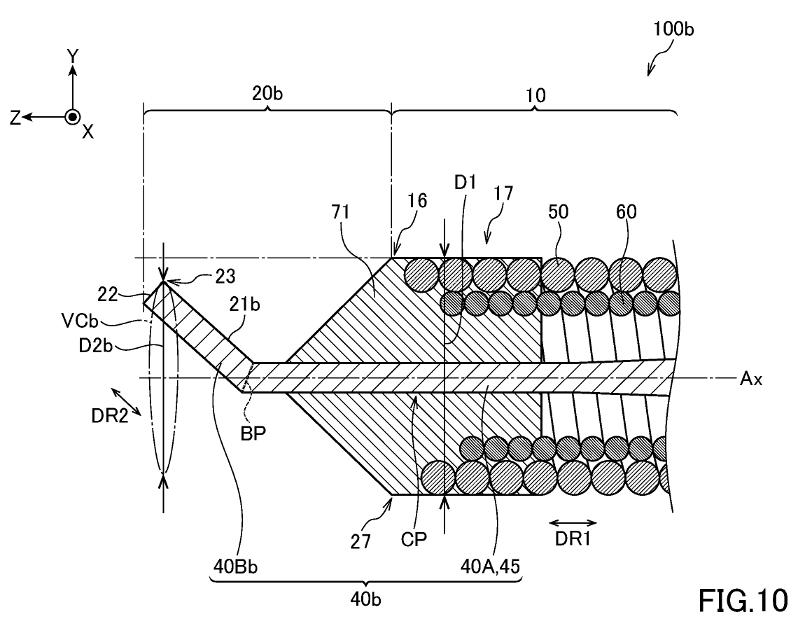

FIG. 10 is an explanatory view schematically illustrating a configuration of a guide wire 100b according to a third embodiment. FIG. 10 illustrates a YZ longitudinal section of a distal end portion of the guide wire 100b. Hereinafter, components of the guide wire 100b according to the third embodiment, which are the same as the components of the guide wire 100 according to the first embodiment, are denoted with the same reference signs, thereby omitting description thereof as appropriate.

In the guide wire 100b of the present embodiment, a distal end 22 of a rod body 21b is located inside an outer peripheral surface 17 of a main body part 10 in a radial direction of the guide wire 100b. When a locus traced by the distal end 22 of the rod body 21b when the guide wire 100b is rotated around a center axis Ax is defined as a virtual circle VCb, a diameter D2b of the virtual circle VCb is smaller than a diameter D1 of the main body part 10. In the radial direction of the guide wire 100b, a distal end of a second portion 40Bb of a core shaft 40b is located inside an outer peripheral surface of a first coil 50.

As described above, in the guide wire 100b of the present embodiment, the distal end 22 of the rod body 21b is located inside the outer peripheral surface 17 of the main body part 10 in the radial direction of the guide wire 100b. According to the guide wire 100b of the present embodiment, since the distal end 22 of the rod body 21b is located inside the outer peripheral surface 17 of the main body part 10, a shape memory property of the guide wire 100b is improved.

### Modifications

The technology disclosed in the present specification is not limited to the embodiments described above, and can be modified into various forms without departing from the gist thereof, and, for example, the following modifications can also be made.

The configuration of the guide wire 100 according to the above-described embodiment is merely an example and can be modified in various ways.

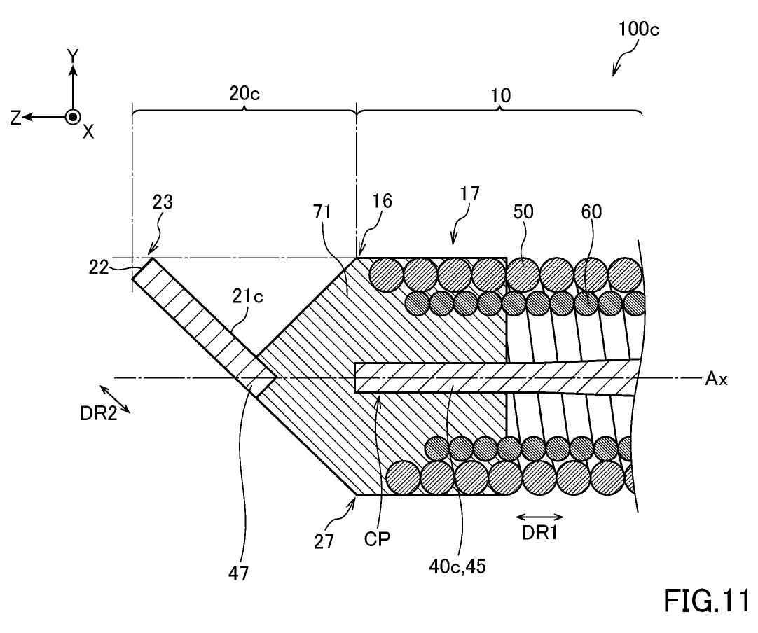

FIG. 11 is an explanatory view schematically illustrating a configuration of a guide wire 100c according to a first modification. FIG. 11 illustrates a YZ longitudinal section of a distal end portion of the guide wire 100c. Hereinafter, components of the guide wire 100c according to the first modification, which are the same as the components of the guide wire 100 according to the first embodiment, are denoted with the same reference signs, thereby omitting description thereof as appropriate.

The guide wire 100c of the present modification includes a core shaft 40c and a distal end wire 47 instead of the core shaft 40 of the first embodiment. A main body part 10 includes the core shaft 40c. A leading portion 20c includes the distal end wire 47. A distal end of the core shaft 40c is embedded in a distal-end-side bonding material 71. A proximal end of the distal end wire 47 is embedded in the distal-end-side bonding material 71. A distal end of the distal end wire 47 coincides with a distal end 22 of a rod body 21c. The distal end wire 47 extends in a second direction DR2. That is, in the guide wire 100c of the present modification, a portion corresponding to the first portion 40A and a portion corresponding to the second portion 40B of the first embodiment are separate bodies. In this manner, the leading portion may be a separate wire from the core shaft.

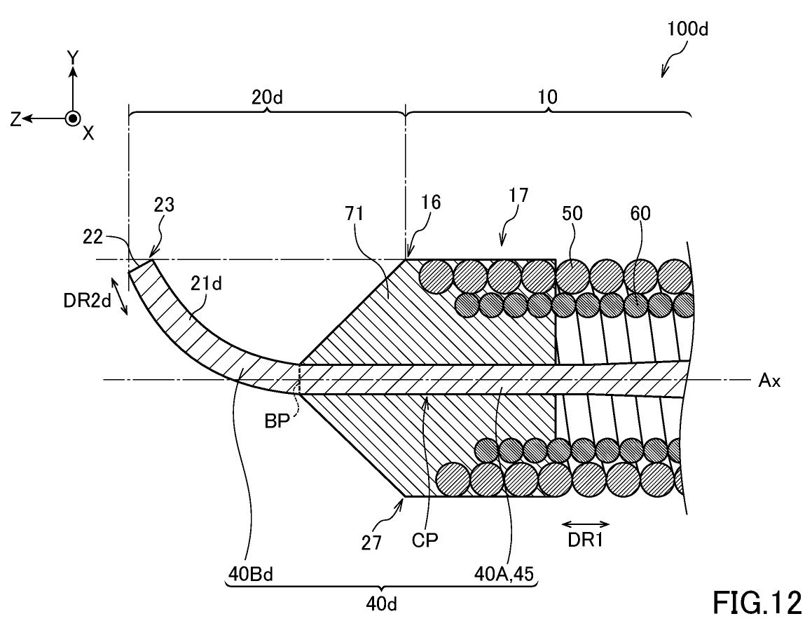

FIG. 12 is an explanatory view schematically illustrating a configuration of a guide wire 100d according to a second modification. FIG. 12 illustrates a YZ longitudinal section of a distal end portion of the guide wire 100d. Hereinafter, components of the guide wire 100d according to the second modification, which are the same as the components of the guide wire 100 according to the first embodiment, are denoted with the same reference signs, thereby omitting description thereof as appropriate.

In the guide wire 100d of the present modification, a rod body 21d in a leading portion 20d extends in a curved shape. A distal end portion of the rod body 21d extends in a second direction DR2d. A second portion 40Bd of a core shaft 40d extends in a curved shape from a boundary position BP with a first portion 40A to a distal end of the second portion 40Bd. A distal end portion of the second portion 40Bd extends in the second direction DR2d. In this manner, the rod body in the leading portion may extend in a curved shape.

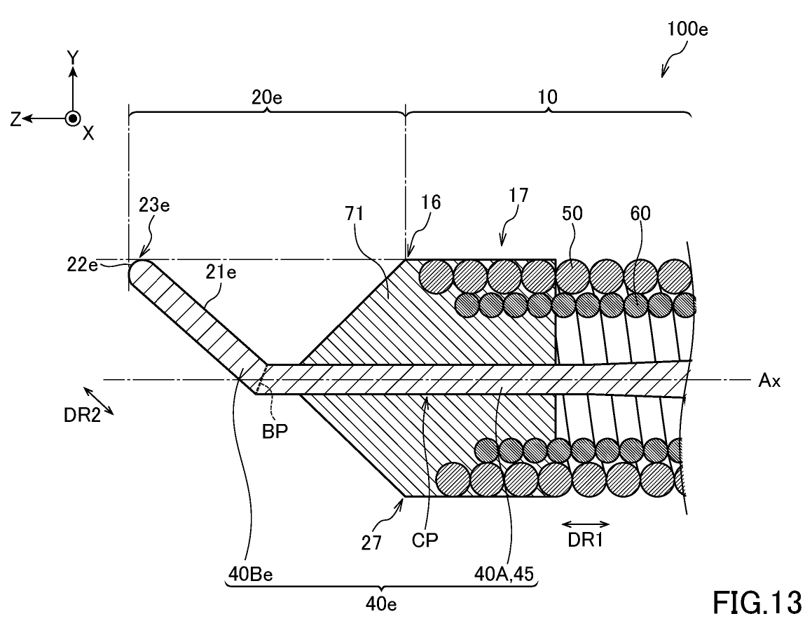

FIG. 13 is an explanatory view schematically illustrating a configuration of a guide wire 100e according to a third modification. FIG. 13 illustrates a YZ longitudinal section of a distal end portion of the guide wire 100e. Hereinafter, components of the guide wire 100e according to the third modification, which are the same as the components of the guide wire 100 according to the first embodiment, are denoted with the same reference signs, thereby omitting description thereof as appropriate.

In the guide wire 100e of the present modification, a distal end 23e of a leading portion 20e is a curved surface. A distal end 22e of a rod body 21e is a curved surface. A distal end of a second portion 40Be of a core shaft 40e is a curved surface. In this manner, the distal end of the leading portion may be a curved surface. A distal end portion of the leading portion may be of any shape. The distal end portion of the leading portion may have, for example, a shape close to a cone that is pointed with an arbitrary position closer to the center than an outer peripheral surface of the leading portion as a distal end. The distal end portion of the leading portion may have, for example, a pointed shape with an arbitrary position on the outer peripheral surface of the leading portion as a distal end and may have a shape including a planar surface extending obliquely with respect to a second direction DR2. The distal end portion of the leading portion may have, for example, an uneven surface. The distal end portion of the leading portion may be, for example, spherical.

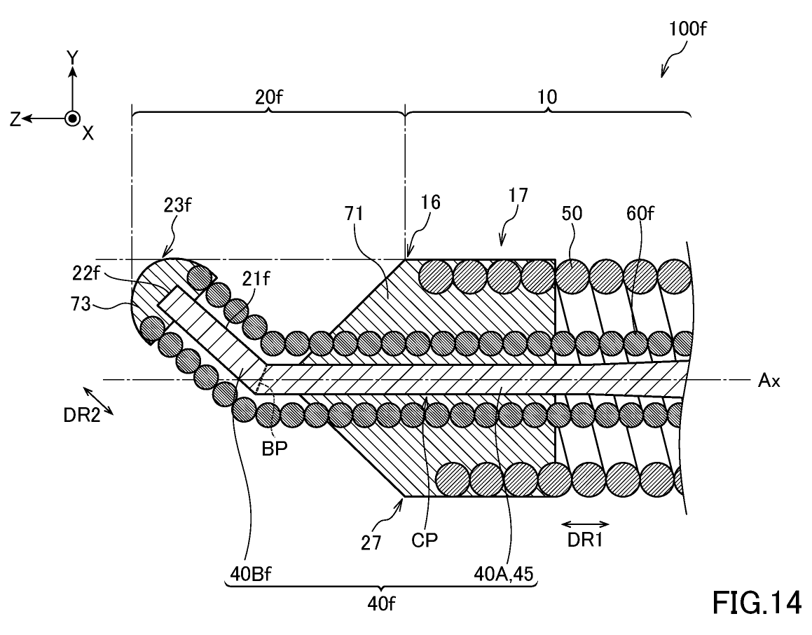

FIG. 14 is an explanatory view schematically illustrating a configuration of a guide wire 100f according to a fourth modification. FIG. 14 illustrates a YZ longitudinal section of a distal end portion of the guide wire 100f. Hereinafter, components of the guide wire 100f according to the fourth modification, which are the same as the components of the guide wire 100 according to the first embodiment, are denoted with the same reference signs, thereby omitting description thereof as appropriate.

In the guide wire 100f of the present modification, a distal end of a second coil 60f covers a distal end portion of a second portion 40Bf of a core shaft 40f. In other words, the distal end of the second coil 60f covers a distal end portion of the portion of the core shaft 40f included in a leading portion 20f. The distal end of the second coil 60f is joined to a distal end of the core shaft 40f via a frontmost end bonding material 73 formed further on the distal end side with respect to a distal-end-side bonding material 71. A surface of the frontmost end bonding material 73 facing the distal end side is a curved surface. The leading portion 20f includes the frontmost end bonding material 73. The frontmost end bonding material 73 is located at a distal end 22f of the leading portion 20f. A distal end 23f of the leading portion 20f is a curved surface. In this manner, the distal end of the second coil may cover the distal end portion of the portion of the core shaft included in the leading portion. The surface of the frontmost end bonding material facing the distal end side may be a curved surface.

The second coil may be a twisted wire including a plurality of strands.

In the longitudinal direction of the medical device, the proximal end of the second portion of the core shaft may be located at a joint part of the core shaft with the tubular body, or may be located on the proximal end side with respect to the joint part of the core shaft with the tubular body. In other words, a boundary position between the first portion and the second portion of the core shaft may be embedded in the distal-end-side bonding material, or may be located on the proximal end side with respect to the distal-end-side bonding material.

The medical device may not necessarily include a second coil.

The transverse section of the second portion of the core shaft may not have a flat shape, and may have any shape such as a circular shape.

The treatment method using the guide wire 100 in the above-described embodiment is merely an example, and can be modified in various ways.

In the above-described embodiment, the guide wire 100 for treating a lesion in a blood vessel has been described as an example. The technology as disclosed herein is similarly applicable to general medical devices for treating a lesion in a living body lumen.

All of the features described in each of the embodiments described above may be appropriately combined with other embodiments, or may be appropriately combined with modifications. All of the features described in each of the modifications described above may be appropriately combined with embodiments, or may be appropriately combined with other modifications. All of the features described in each of the embodiments described above may be omitted as appropriate. All of the features described in each of the modifications described above may be omitted as appropriate.

1. A medical device comprising: an elongated main body part; and
a leading portion connected to a distal end of the main body part, the leading portion comprising a rod body that is configured to enter a lesion, wherein
a distal end portion of the main body part extends in a first direction, and
a distal end portion of the rod body extends in a second direction different from the first direction.

2. The medical device according to claim 1, wherein
in a radial direction of the medical device, a distal end of the rod body is located at the same position as an outer peripheral surface of the main body part.

3. The medical device according to claim 1, wherein
in a radial direction of the medical device, a distal end of the rod body is located outside an outer peripheral surface of the main body part.

4. The medical device according to claim 1, wherein
in a radial direction of the medical device, a distal end of the rod body is located inside an outer peripheral surface of the main body part.

5. The medical device according to claim 1, further comprising a core shaft, wherein
the main body part includes a first portion of the core shaft, the first portion including a distal end portion extending in the first direction, and
the leading portion includes a second portion of the core shaft and that is different from the first portion, the second portion being connected to a distal end of the first portion and extending in the second direction.

6. The medical device according to claim 5, wherein
the main body part further includes a tubular body that covers the core shaft and that is joined to the core shaft.

7. The medical device according to claim 6, wherein
a proximal end of the second portion is located on a distal end side with respect to a joint part of the core shaft at which the core shaft is joined with the tubular body in a longitudinal direction of the medical device.

8. The medical device according to claim 6, wherein
the tubular body is a first coil, and
the medical device further comprises a second coil located between the core shaft and the first coil in a radial direction of the medical device.

9. The medical device according to claim 5, wherein
a transverse section of the second portion has a flat shape
